(19) United States
(12) Patent Application Publication
(10) Pub. No.: US 2007/0161791 A1
(43) Pub. Date: Jul. 12, 2007

(54) Process for the preparation of terazosin hydrocloride dihydrate

(76) Inventors: Narsimha Reddy Penthala, Hyderabad (IN); Anand Gopalkrishna Kamat, Hyderabad (IN); Sivakumaran Meenakshisunderam, Hyderabad (IN)

Correspondence Address: Jay R. Akhave;# 612, 2058 N Mills Ave, Claremont, CA, 91711, US

(21) Appl. No.: 11/649,053

(22) Filed: Jan. 3, 2007

(30) Foreign Application Priority Data
Jan. 9, 2006 (IN) 37/CHE/2006

(51) Int. Cl. C07D 405/14

(52) U.S. Cl. 544/284

(57) ABSTRACT

The present invention relates to an improved process for the preparation of 1-(4-amino-6,7-dimethoxyquinazolin-2-yl)-4-[[(2RS)-2,3,4,5-tetrahydrofuran-2-yl]carbonyl]piperazine hydrochloride dihydrate of Formula I, through an intermediate 1-(4-amino-6,7-dimethoxyquinazolin-2-yl)-4-[[(2RS)-2,3,4,5-tetrahydrofuran-2-yl]carbonyl]piperazine hydrochloride/hydrobromide (V).

### CROSS REFERENCE TO THE RELATED APPLICATION

This application claims the priority of an Indian Patent application No. 37/CHE/2006 filed on Jan. 9, 2006.

| NAME | RESIDENCE | CITIZENSHIP |
|---|---|---|
| Narsimha | 2-22/1/138, | India |
| Reddy Penthala | Simhadri Towers | |
| | Flat No. 103 | |
| | Madhavi Nagar, | |
| | Kukatpally | |
| | Hyderabad-500 072 | |
| | India | |
| Anand | 101, Home Sree Towers | India |
| Gopalkrishna Kamat | Near Hanuman Temple | |
| | Jayanagar, Kukatpally | |
| | Hyderabad-500 072 | |
| | India | |
| Sivakumaran | D-1, Hidden Treasure Apts | India |
| Meenakshisunderam | Near Ayyappa Swami Temple | |
| | Lane Somajiguda, | |
| | Hyderabad-500 082 | |
| | India | |

### FIELD OF THE INVENTION

The present invention relates to an improved process for the preparation of 1-(4-amino-6,7-dimethoxyquinazolin-2-yl)-4-[[(2RS)2,3,4,5-tetrahydrofuran-2-yl]carbonyl]piperazine of Formula I

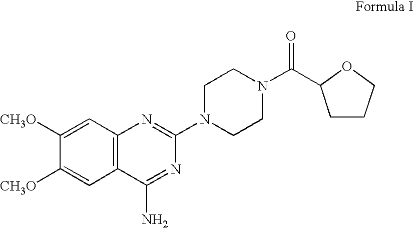

### BACKGROUND OF THE INVENTION

1-(4-Amino-6,7-dimethoxyquinazolin-2-yl)-4-[[(2RS)-2,3,4,5-tetrahydrofuran-2-yl]carbonyl]piperazine, generically known as Terazosin, is used for the treatment of hypertension and benign prostatic hyperplasia.

Abbott Laboratories has disclosed Terazosin and its pharmaceutically acceptable salts in U.S. Pat. No. 4,026,894. This patent also describes a process for the preparation of Terazosin, which involves reaction of 4-amino-2-chloro-6,7-dimethoxyquinazoline with 1-[[(2RS)-2,3,4,5-tetrahydrofuran-2-yl]carbonyl]piperazine of Formula II in methoxyethanol. Terazosin base is then converted to hydrochloride salt in methanol using hydrogen chloride solution in isopropanol.

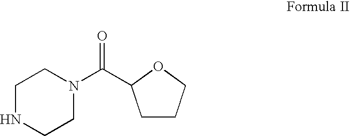

Terazosin hydrochloride has been reported to exist in several crystalline Polymorphic Forms including non-solvated crystalline Forms such as Form I, Form II, Form III, Form IV, methanolate Form, Monohydrate crystalline Form and Dihydrate crystalline Form.

U.S. Pat. No. 4,251,532 describes a crystalline dihydrate Form of Terazosin hydrochloride and states that dihydrate Form of Terazosin hydrochloride is more stable during storage than the anhydrous Form. This dihydrate Form of Terazosin hydrochloride has been marketed all over the world with the trade name HYTRIN. Further, this patent also describes a process for the preparation of Terazosin hydrochloride dihydrate, which involves as an initial step, the preparation of Terazosin base by reacting 4-amino-2-chloro-6,7-dimethoxyquinazoline with 1-[[(2RS)-2,3,4,5-tetrahydrofuran-2-yl]carbonyl]piperazine of Formula II in a suitable solvent such as ethyleneglycol, monomethyl ether, methoxy ethanol in the presence of triethylamine base. In the subsequent step, Terazosin base is converted to its hydrochloride dihydrate salt by treating with hydrochloric acid

U.S. Pat. No. 5,504,207 also relates to a process for the preparation of Terazosin hydrochloride dihydrate. The disclosed process involves as an initial step in which Terazosin hydrochloride Form IV is first prepared by reacting 4-amino-2-chloro-6,7-dimethoxyquinazoline with 1-[[(2RS)-2,3,4,5-tetrahydrofuran-2-yl]carbonyl]piperazine in the absence of an acid scavenger. The Form IV is then converted to Terazosin hydrochloride dihydrate.

U.S. Pat. No. 6,248,888 further relates to a process for the preparation of 1-(4-amino-6,7-dimethoxyquinazolin-2-yl)4-[[(2RS)3-2,3,4,5-tetrahydrofuran-2-yl]carbonyl]piperazine (Terazosin) hydrochloride dihydrate, which comprises heating 4-amino-2-chloro-6,7-dimethoxyquinazoline and 1-[[(2RS)-2,3,4,5-tetrahydrofuran-2-yl]carbonyl]piperazine in a polar organic reaction solution, wherein said polar organic reaction solution comprises a polar organic solvent and a minimum amount of added water effective to precipitate out the said 1-(4-amino-6,7-dimethoxyquinazolin-2-yl)-4-[[(2RS)-2,3,4,5-tetrahydrofuran-2-yl]carbonyl]piperazine hydrochloride dihydrate.

Canadian Pat. No. 2,150,985 describes a process for preparing Terazosin hydrochloride dihydrate, which initially involves the preparation of Terazosin base by reacting N-(3,4-dimethoxy-6-cyanoaniline)-1-yl-formamide and 1-[[(2RS)-2,3,4,5-tetrahydrofuran-2-yl]carbonyl]piperazine in presence of pyridine followed by treatment with phosphorus oxychloride. Subsequently, the addition of hydrochloric acid to the suspension of Terazosin base in water results in formation of Terazosin hydrochloride dihydrate.

The known methods, discussed herein above, involve reaction of 4-amino-2-chloro-6,7-dimethoxyquinazoline with 1-[[(2RS)-2,3,4,5-tetrahydrofuran-2-yl]carbonyl]piperazine in the presence or absence of an acid scavenger in a suitable solvent to produce Terazosin (I). Major disadvantage of the above mentioned processes is that 1-[[(2RS)-2,3,4,5-tetrahydrofuran-2-yl]carbonyl]piperazine (II) invariably contains 1-[(furan-2-yl)carbonyl]piperazine (III)

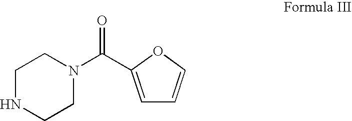

as an impurity, which results in corresponding amount of Prazosin (IV) as impurity in Terazosin.

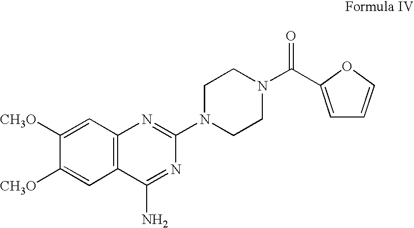

Removal of Prazosin has often proved to be difficult due to its structural similarity with Terazosin. Furthermore, 1-[[(2RS)-2,3,4,5-tetrahydrofuran-2-yl]carbonyl]piperazine (II) is an oily material and cannot be purified to remove 1-[(furan-2-yl)carbonyl]piperazine(III) impurity to meet the USP limit of Prazosin ‘Not More Than 0.1%’ in Terazosin hydrochloride finished product.

In the process of the present invention, we have now found that 1-[[(2RS)-2,3,4,5-tetrahydrofuran-2-yl]carbonyl]piperazine (II) can be purified by making its crystalline acid addition salt, which is used as such to produce Terazosin of high purity.

### OBJECTIVE OF INVENTION

The main objective of the present invention is to provide a simple and effective process for the preparation of Terazosin of high purity on commercial scale.

### SUMMARY OF THE INVENTION

Accordingly, the present invention provides a method for the preparation of Terazosin from 1-[[(2RS)-2,3,4,5-tetrahydrofuran-2-yl)carbonyl]piperazine (II), through an intermediate 1-(4-amino-6,7-dimethoxyquinazolin-2-yl)-4-[[(2RS)-2,3,4,5-tetrahydrofuran-2-yl]carbonyl]piperazine hydrochloride/hydrobromide (VI), which comprises:
i) reaction of 1-[[(2RS)-2,3,4,5-tetrahydrofuran-2-yl]carbonyl]piperazine (II), with hydrobromic acid in an organic solvent to give 1-[[(2RS)-2,3,4,5-tetrahydrofuran-2-yl]carbonyl]piperazine hydrobromide (IIa),
ii) condensation of compound (IIa) in presence of a base with 4-amino-2-chloro-6,7-dimethoxyquinazoline (V) in an organic solvent to produce the intermediate, 1-(4-amino-6,7-dimethoxyquinazolin-2-yl)-4-[[(2RS)-2,3,4,5-tetrahydrofuran-2-yl]carbonyl]piperazine hydrochloride/ hydrobromide (VI),
iii) extracting compound (VI) into water and adding inorganic base to produce 1-(4-amino-6,7-dimethoxyquinazolin-2-yl)4-[[(2RS)-2,3,4,5-tetrahydrofuran-2-yl]carbonyl]piperazine (Terazosin) (I).

The reaction scheme is given below:

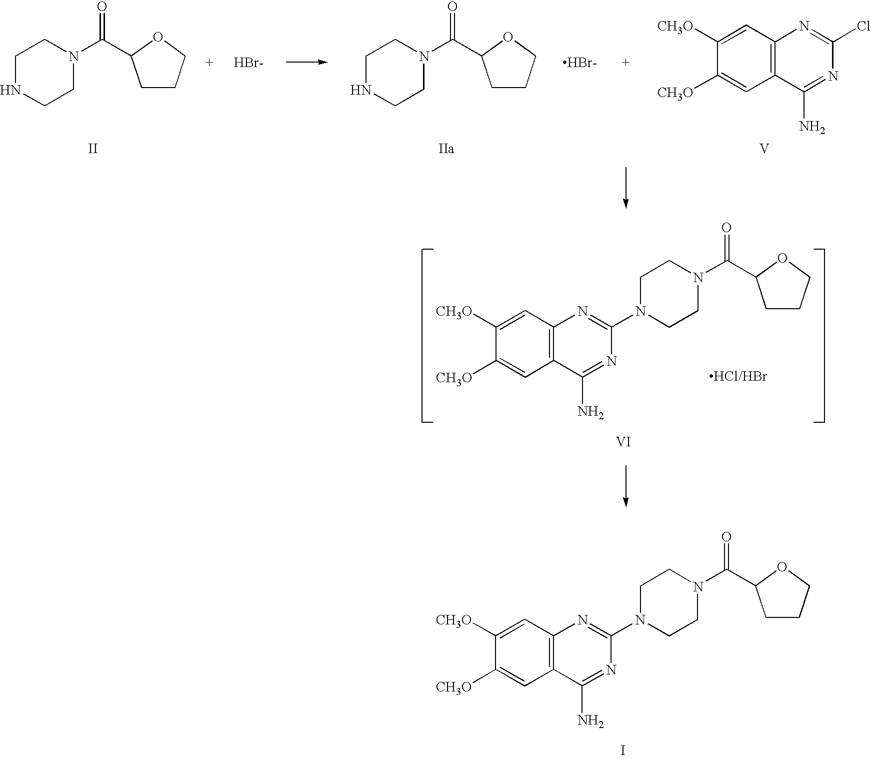

### DETAILED DESCRIPTION OF THE INVENTION

1-[[(2RS)-2,3,4,5-tetrahydrofuran-2-yl]carbonyl]piperazine (II) is treated with a molar equivalent of hydrobromic acid in an organic solvent selected from alcohols such as ethanol, n-propanol, isopropanol, n-butanol, isobutanol or mixtures thereof, preferably n-butanol, at a temperature of about 0-50° C. The product obtained is isolated by filtration and dried to give 1-[[(2RS)-2,3,4,5-tetrahydrofuran-2-yl]carbonyl]piperazine hydrobromide (IIa). It has been observed that preparation of hydrobromide salt of Formula (II) results in removal of 1-[(furan-2-yl)carbonyl]piperazine (III) impurity to less than 0.1% by HPLC analysis.

Condensation of compound (IIa) with 4-amino-2-chloro-6,7-dimethoxyquinazoline (V) is carried out in presence of a base selected from 1,8-Diazabicyclo[5.4.0]undecane-7-ene, triethylamine, N,N-diisopropylethylamine, preferably triethylamine in a water immiscible alcohol. The water immiscible alcohols are selected from n-butanol, isobutanol, amyl alcohol, isoamyl alcohol, preferably n-butanol. The reaction mass is heated to a temperature of about 100° C. to 140° C., most preferably at about 115-120° C. for about 2-5 hrs. Thereafter, reaction mass is cooled to about 50° C. and is extracted with water. The aqueous extract containing 1-(4-amino-6,7-dimethoxyquinazolin-2-yl)-4-[[(2RS)-2,3,4,5-tetrahydrofuran-2-yl]carbonyl]piperazine hydrochloride/hydrobromide (VI) is treated with an inorganic base selected from sodium hydroxide, potassium hydroxide, aqueous ammonia, most preferably aqueous ammonia, until the pH is about 8.0-10.0 at a temperature of about 30-55° C., most preferably at 40-45° C. The slurry is cooled to 25-30° C. for about 15 minutes to 1 hr and 1(4-amino-6,7-dimethoxyquinazolin-2-yl)4-[[(2RS)-2,3,4,5-tetrahydrofuran-2-yl]carbonyl]piperazine (Terazosin) (I) is collected by filtration and dried.

Terazosin (I) is converted to Terazosin hydrochloride dihydrate by conventional methods reported in the prior-art. Terazosin (I) is added to a mixture of water and ethanol at 60-65° C. Concentrated hydrochloric acid is added to the slurry. The solution thus obtained is treated with carbon and Terazosin hydrochloride dihydrate is crystallized by cooling to 0-5° C.

The following examples illustrate the nature of the invention and are provided for illustrative purposes only and should not be construed to limit the scope of the invention.

### Stage-I

### EXAMPLE 1

### Preparation of 1-[((2R-2,3,4,5tetrahydrofuran-2-yl]carbonyl]piperazine hydrobromide (Tetrahydrofuroyl piperazine hydrobromide)

1-[[(2RS)-2,3,4,5-tetrahydrofuran-2-yl]carbonyl]piperazine (600 g, 3.26 mol) of HPLC purity 96.35% and containing 0.32% of 1-[(furan-2-yl)carbonyl]piperazine impurity was dissolved in 1-butanol (3600 ml). Hydrobromic acid (535.76 g, 49.3% w/w) was added slowly at 20-40° C. The contents were cooled to 5-10° C. slowly in 1 hr and continued stirring at this temperature for further 30 min. The product was filtered, washed with pre-cooled 1-butanol (2×600 ml, 5-10° C.) and dried at 40-45° C. under reduced pressure to obtain 840 g of 1-[[(2RS)-2,3,4,5-tetrahydrofuran-2-yl]carbonyl]piperazine hydrobromide having 99.42% purity and contains 0.03% of 1-[(furan-2-yl)carbonyl]piperazine impurity (HPLC).

### EXAMPLE 2

### Preparation of 1-[[(2RS)2,3,4,5-tetrahydrofuran-2-yl]carbonyl]piperazine hydrobromide (Tetrahydrofuroyl piperazine hydrobromide)

1-[[(2RS)-2,3,4,5-tetrahydrofuran-2-yl]carbonyl]piperazine (10 g, 0.054 mol) of HPLC purity 90.52% and containing 0.61% of 1-[(furan-2-yl)carbonyl]piperazine impurity was dissolved in 1-butanol (60 ml). Hydrobromic acid (8.92 g, 49.3% w/w) was added slowly at 20-40° C. The contents were cooled to 5-10° C. slowly in 1 hr and continued stirring at this temperature for further 30 min. The product was filtered, washed with pre-cooled 1-butanol (2×10 ml, 5-10° C.) and dried at 40-45° C. under reduced pressure to obtain 13.25 g of 1-[[(2RS)-2,3,4,5-tetrahydrofuran-2-yl]carbonyl]piperazine hydrobromide having 99.07% purity and contains 0.04% of 1-[(furan-2-yl)carbonyl]piperazine impurity (HPLC).

### Stage II

### Preparation of 1-(4-amino-6,7-dimethoxyquinazolin-2-yl)-4-[[(2RS)-2,3,4,5-tetrahydrofuran-2-yl]carbonyl]piperazine (Terazosin base)

A mixture of 4-amino-2-chloro-6,7-dimethoxyquinazoline (60 g, 0.25 mol), 1-[[(2RS)-2,3,4,5-tetrahydrofuran-2-yl]carbonyl]piperazine hydrobromide (73 g, 0.275 mol), triethylamine (26.30 g, 0.26 mol) and 1-butanol (360 ml) was heated at 115-120° C. for 3 hrs. Reaction mass was cooled to 50° C., water (600 ml, 25° C.) was added and stirred at 30-35° C. for 15 min. The reaction mass was cooled to 5-10° C. for 15 min. Thereafter, it was filtered and residue was washed with pre-cooled DM water (2×60 ml, 5-10° C.). The lower aqueous layer was separated from the filtrate and organic layer was extracted with DM water (120 ml). The combined aqueous extract was basified to pH 8.4 at 40-45° C. with aqueous ammonia (35 ml, 18% w/w) and stirring was continued at this temperature for 30 min. Thereafter, the slurry was cooled to 25-35° C. and stirred for 30 min. The product was filtered, washed with DM water (2×120 ml, 25-30° C.) and dried at 45-50° C. under reduced pressure to obtain 89.5 g of the title compound having purity 99.82% and Prazosin impurity 0.03% (HPLC).

### Stage III

### Preparation of 1-(4-amino6,7-dimethoxyquinazolin-2-yl)-4[[(2RS)-2,3,4,5-tetrahydrofuran-2-yl]carbonyl]piperazine hydrochloride (Terazosin hydrochloride)

A mixture of Terazosin base (80 g, 0.207 mol), ethanol (1106 ml), DM water (94 ml) was heated to 60-65° C. and hydrochloric acid (20.50 g, 36.8% w/w) was added at 60-65° C. The resulting clear solution was treated with carbon (2.40 g) at 60-65° C. for 15 min. Carbon was removed by filtration and the residue was washed with preheated 8% v/v aqueous ethanol (2×80 ml, 60-65° C.). The filtrate was cooled and stirred at 0-5° C. for 4 hrs. The product was filtered, washed with precooled 8% v/v aqueous ethanol (2×80 ml, 0-5° C.) and dried at 45-50° C. under reduced pressure to obtain 85 g of Terazosin hydrochloride dihydrate, having purity 99.87% and Prazosin 0.02% (HPLC).

We claim:

1. An improved process for the preparation of 1-(4-amino-6,7-dimethoxyquinazolin-2-yl)-4-[[(2RS)-2,3,4,5-tetrahydrofuran-2-yl]carbonyl]piperazine of Formula (I) 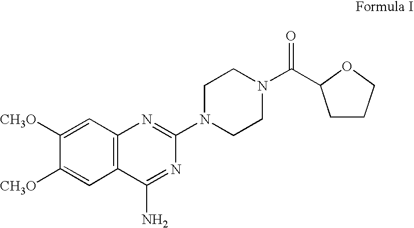
which comprises treating 1-(4-amino-6,7-dimethoxyquinazolin-2-yl)4-[[(2RS)-2,3,4,5-tetrahydrofuran-2-yl]carbonyl]piperazine hydrochloride/hydrobromide (VI) with an inorganic base to precipitate the compound Formula I.

2. The process according to claim 1, wherein the inorganic base used is selected from aqueous ammonia, aqueous sodium hydroxide and aqueous potassium hydroxide.

3. The process according to claim 2, wherein the inorganic base used is aqueous ammonia.

4. The process according to claim 1, wherein the compound of Formula (I) is converted into 1-(4-amino-6,7-dimethoxyquinazolin-2-yl)-4-[[(2RS)-2,3,4,5-tetrahydrofuran-2-yl]carbonyl]piperazine hydrochloride dihydrate.

5. An improved process for the preparation of 1-(4-amino-6,7-dimethoxyquinazolin-2-yl)-4-[[(2RS)-2,3,4,5-tetrahydrofuran-2-yl]carbonyl]piperazine of Formula (I) 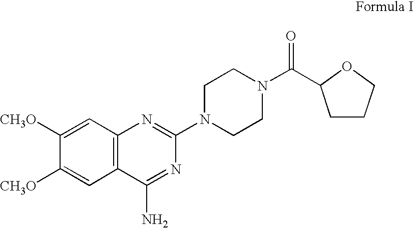
which comprises:
i) condensing 1-[[(2RS)-2,3,4,5-tetrahydrofuran-2yl]carbonyl]piperazine hydrobromide (IIa) 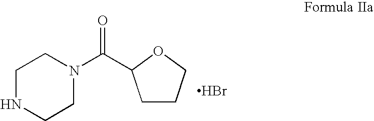
in presence of a base with 4-amino-2-chloro-6,7-dimethoxyquinazoline (V), 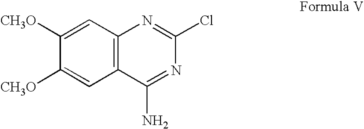
in an organic solvent to produce the intermediate, 1-(4-amino-6,7-dimethoxyquinazolin-2-yl)4-[[(2RS)-2,3,4,5-tetrahydrofuran-2-yl]carbonyl]piperazine hydrochloride/hydrobromide (VI), 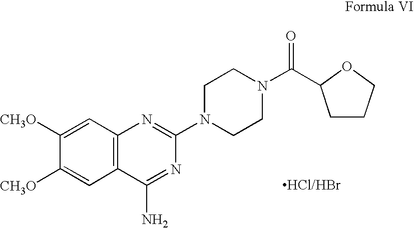
ii) treating the aqueous solution of compound of Formula (VI) with an inorganic base to precipitate the compound to Formula I.

6. The process according to claim 5, wherein the base used in condensation step (step-i) is selected from 1,8-diazabicyclo[5.4.0]undecane-7-ene, triethylamine and N,N-diisopropylethylamine.

7. The process according to claim 6, wherein the base is triethylamine.

8. The process according to claim 5, wherein the organic solvent used in (step-i) is a water immiscible alcohol selected from n-butanol, isobutanol, amyl alcohol, isoamyl alcohol.

9. The process according to claim 8, wherein the solvent is n-butanol.

10. The process according to claim 5, wherein the inorganic base used in (step-ii) is selected from aqueous ammonia, aqueous sodium hydroxide and aqueous potassium hydroxide.

11. The process according to claim 10, wherein the inorganic base used is aqueous ammonia.

12. The process according to claim 5, wherein the compound of Formula (I) is converted into 1-(4-amino-6,7-dimethoxyquinazolin-2-yl)-4-[[(2RS)-2,3,4,5-tetrahydrofuran-2-yl]carbonyl]piperazine hydrochloride dihydrate.

13. An improved process for the preparation of 1-(4-amino-6,7-dimethoxyquinazolin-2-yl)-4-[[(2RS)-2,3,4,5-tetrahydrofuran-2-yl]carbonyl]piperazine of Formula (I) 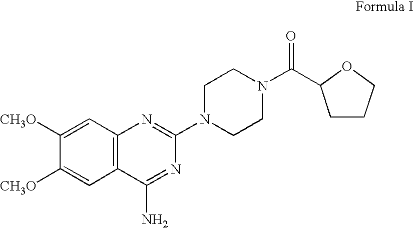
which comprises:
i) condensing 1-[[(2RS)-2,3,4,5-tetrahydrofuran-2yl]carbonyl]piperazine hydrobromide (IIa) 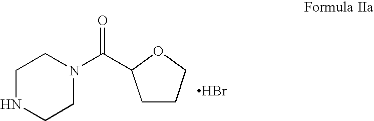
in presence of a base with 4-amino-2-chloro-6,7-dimethoxyquinazoline (V), 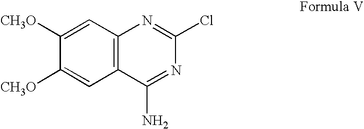
in an organic solvent to produce the intermediate, 1-(4-amino-6,7-dimethoxyquinazolin-2-yl)4-[[(2RS)-2,3,4,5-tetrahydrofuran-2-1]carbonyl]piperazine hydrochloride/hydrobromide (VI), 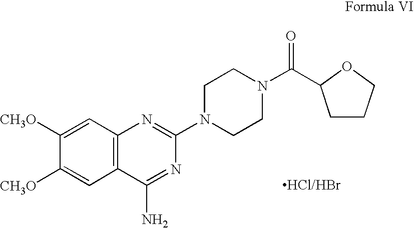
ii) extracting compound (VI) into water and adding inorganic base to the aqueous extract to isolate 1-(4-amino-6,7-dimethoxyquinazolin-2-yl)4-[[(2RS)-2,3,4,5-tetrahydro furan-2-yl]carbonyl]piperazine (Terazosin) (I).

14. The process according to claim 13, wherein the base used in condensation step (step-i) is selected from 1,8-diazabicyclo[5.4.0]undecane-7-ene, triethylamine and N,N-diisopropylethylamine.

15. The process according to claim 14, wherein the base used is triethylamine.

16. The process according to claim 13, wherein the organic solvent used in (step-i) is a water immiscible alcohol selected from n-butanol, isobutanol, amyl alcohol, isoamyl alcohol.

17. The process according to claim 16, wherein the solvent is n-butanol.

18. The process according to claim 13, wherein the inorganic base used in (step-ii) is selected from aqueous ammonia, aqueous sodium hydroxide and aqueous potassium hydroxide.

19. The process according to claim 18, wherein the inorganic base used is aqueous ammonia.

20. The process according to claim 13, wherein the compound of Formula (I) is converted into 1-(4-amino-6,7-dimethoxyquinazolin-2-yl)4-[[(2RS)-2,3,4,5-tetrahydrofuran-2-yl]carbonyl]piperazine hydrochloride dihydrate.

21. A process for the preparation of 1-(4-amino-6,7-dimethoxyquinazolin-2-yl)-4-[[(2RS)-2,3,4,5-tetrahydrofuran-2-yl]carbonyl]piperazine of Formula (I) 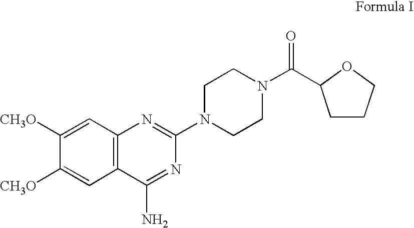
substantially free of compound of Formula (IV) having the following structure 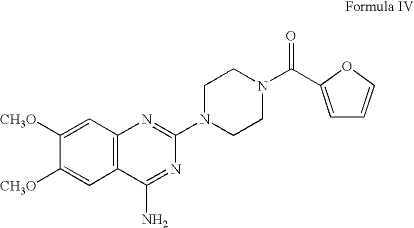
by using for a starting material a hydrobromide salt of the compound of Formula (II).
